(19) United States
(12) Patent Application Publication
(10) Pub. No.: US 2024/0110190 A1
(43) Pub. Date: Apr. 4, 2024

(54) COMPOSITIONS AND METHODS FOR TREATING PULMONARY ARTERIAL HYPERTENSION (PAH) AND OTHER DISORDERS

(71) Applicant: THE TRUSTEES OF THE UNIVERSITY OF PENNSYLVANIA, Philadelphia, PA (US)

(72) Inventors: Yoseph BARASH, Wynnewood, PA (US); David Sheng Ming LEE, Philadelphia, PA (US); Louis R. GHANEM, Villanova, PA (US); Nicholas J. HAND, Glenside, PA (US)

(73) Assignee: THE TRUSTEES OF THE UNIVERSITY OF PENNSYLVANIA, Philadelphia, PA (US)

(21) Appl. No.: 18/264,885

(22) PCT Filed: Feb. 28, 2022
(86) PCT No.: PCT/US2022/018140

§ 371 Date: Aug. 9, 2023

Related U.S. Application Data
(60) Provisional application No. 63/154,313, filed on Feb. 26, 2021.

(51) Int. Cl. C12N 15/113

(52) U.S. Cl. CPC C12N 15/1138; C12N 2310/11; C12N 2310/315; C12N 2310/321; C12N 2310/3231

(57) ABSTRACT

Compositions and methods for treating fibrosis using oligonucleotide-based therapies are provided. These therapies can be used to increase expression of Bone morphogenetic protein receptor type II (BMPR2) via inhibition of upstream Open Reading Frames, thereby reducing vasoconstriction, vascular remodeling, and formation of fibrosis, particularly pulmonary arterial fibrosis related in patients in need thereof.

### CROSS REFERENCE TO RELATED APPLICATION

This application claims priority of U.S. Provisional application No. 63/154,313 filed Feb. 26, 2021, the entire contents being incorporated herein by reference as though set forth in full.

### STATEMENT OF GRANT SUPPORT

This invention was made with government support under Grant number GM128096 awarded by the National Institutes of Health. The government has certain rights in the invention.

### INCORPORATION-BY-REFERENCE OF MATERIAL SUBMITTED IN ELECTRONIC FORM

Incorporated herein by reference in its entirety is the Sequence Listing submitted via EFS-Web as a text file named SEQLIST.txt, created Feb. 28, 2022 and having a size of 8,192 bytes.

### FIELD OF THE INVENTION

The present invention relates to compositions and methods, more particularly to antisense oligonucleotides for treating PAH associated pathophysiology, vasoconstriction, aberrant vascular remodeling and fibrosis, thereby ameliorating symptoms thereof. The antisense oligonucleotides may also be used to modulate BMPR2 signaling for treatment of other pathological conditions associated with BMPR2 signaling pathways.

### BACKGROUND OF THE INVENTION

Several publications and patent documents are cited throughout the specification in order to describe the state of the art to which this invention pertains. Each of these citations is incorporated herein by reference as though set forth in full.

Bone morphogenetic protein receptor type II (BMPR2) is a serine/threonine receptor kinase that has been implicated in diverse cellular functions and human diseases. As a member of the TGFβ receptor superfamily, BMPR2 functions by forming heterotetrameric receptor signaling complexes with associated Type I receptors including ALK1/2/3/6. Receptor activation by endogenous bone morphogenetic peptide (BMP) ligands induces downstream phosphorylation of SMAD1/5/8 proteins which control numerous downstream transcriptional responses to BMP stimulation.

BMPR2 is a known haploinsufficient gene, and heterozygous loss-of-function mutations in BMPR2 lead to familial pulmonary arterial hypertension (PAH). Moreover, in patients with acquired, or idiopathic pulmonary arterial hypertension, BMPR2 expression is often reduced compared to normal healthy controls. Thus, increasing activity through the BMPR2 signaling pathway has long been identified as a key therapeutic goal in the treatment of both familial and idiopathic forms of PAH and pulmonary hypertension more generally.

PAH is a deadly disease characterized by progressive vascular remodeling of pulmonary arteries and arterioles leading to hyperplasia, hypertrophy, fibrosis, and in situ thrombi formation in small pulmonary arteries and arterioles for which the only curative treatment is lung transplantation. Approximately 75-80% of familial and 11-40% of sporadic PAH patients carry loss-of-function mutations in the BMPR2 receptor [1]. Mutation carriers are known to have worse prognosis compared to non-carriers [2], and the penetrance of PAH in mutation carriers has been linked to expression of the normal BMPR2 allele [3]. Loss of BMPR2 signaling has been shown to promote morphological changes in pulmonary arterial endothelial cells leading to their trans-differentiation into more mesenchymal phenotypes associated with increased expression of smooth muscle cell markers, breakdown of endothelial junctions, acquisition of spindle-shaped morphologies, and loss of endothelial gene expression markers [4]. Insufficiency of BMPR2 has also been shown to heighten intracellular inflammatory responses in pulmonary artery endothelial cells [5]. Moreover, BMPR2 expression is often reduced compared to normal healthy controls in patients with acquired or idiopathic PAH in the absence of germline loss-of-function BMPR2 mutations [6,7].

Given the central role of BMPR2 dose in the pathogenesis of PAH, identifying methods to increase signaling activity through the BMPR2 axis has been a longstanding goal for the treatment of both familial and idiopathic forms of PAH [8]. Several strategies have been proposed to indirectly increase BMPR2 signaling therapeutically in patients with pulmonary arterial hypertension—but most of these are non-targeted and can have significant toxicities. A Phase II clinical trial of Tacrolimus (which was identified in a drug screen to non-specifically increase downstream BMPR2 signaling) was ineffective because of the inherent toxicities associated with Tacrolimus treatment.

Antisense oligonucleotides are an emerging class of therapeutic molecules that can modulate post-transcriptional gene expression by targeting mRNA sequences specifically. Examples of ASO-based therapies currently in clinical use include those having efficacy for retinitis associated with CMV infection, homozygous familial hypercholesterolemia, spinal muscular atrophy, and cancer. While these therapeutic approaches using ASOs have traditionally targeted mRNAs for degradation, there is emerging interest in the ability for ASOs to selectively increase endogenous protein expression. Because all mRNAs encode 5′UTRs, targeting translational regulatory elements in 5′UTRs represents a promising therapeutic application of ASO technology. The promise of using ASOs to block uORFs in 5′UTRs heretofore has been limited by incomplete annotation of regulatory uORFs in mRNA 5′UTRs.

### SUMMARY OF THE INVENTION

Pulmonary arterial hypertension (PAH) is a condition defined by abnormalities in vessel functionality and responses to stressors which culminate in aberrant growth of endothelial cells (ECs) and smooth muscle cells (SMCs), leading to vascular obstruction and the formation of plexiform lesions. The increased pulmonary vascular resistance enhances the load upon the right ventricle (RV). The RV will compensate with hypertrophy, which progresses to RV-failure and death. Current available therapies for PAH mainly target vasoconstriction to reduce pressures and relieve the load, with some showing anti-proliferative effects in vitro. These drugs decelerate, but do not stop disease progression. As noted above, BMPR2 signaling is associated with the pathological phenotypic changes observed in PAH and other forms of fibrosis.

Thus, in accordance with the present invention, a composition for enhancing expression of bone morphogenetic protein receptor type II (BMPR2) is disclosed comprising at least one antisense oligonucleotide (ASO) which may or may not comprise modifications which increase stability in bodily fluids. In certain embodiments, the ASO targets at least one of uORF-6, uORF-9, or both in the 5′UTR of the BMPR2 gene. In particularly preferred embodiments, the ASO targets an upstream Open Reading Frame (uORF) and comprises a nucleic acid of SEQ ID NO: 28, 30, 31, or 32. In certain aspects the ASOs are present in a vector.

The compositions of the invention may be formulated for administration selected from oral administration, aerosol administration, parenteral administration, intravenous administration or intracardiac administration. In certain approaches, the ASOs are incorporated into a nanoparticle formulation suitable for aerosolized administration. In alternative approaches, the ASOs are incorporated into a formulation suitable for intracardiac administration.

The invention also provides methods of treating PAH-associated pathophysiology including, without limitation, increased vasoconstriction, aberrant vascular remodeling and fibrosis in a subject in need thereof comprising administration of and effective amount of the composition in any one of the preceding claims, said administration being effective to reduce fibrosis in said subject. In certain embodiments, the fibrosis is selected from pulmonary fibrosis, cardiac fibrosis, or liver fibrosis. In preferred embodiments, the ASOs modulate BMPR2 signaling. In preferred embodiments, the fibrosis is caused by pulmonary arterial fibrosis (PAH) and said treatment with said ASOs reduces vasoconstriction and vascular remodeling. The subject in need thereof can have PAH-related symptoms. These include, without limitation, increased shortness of breath, fatigue, edema, swelling of the feet, legs and eventually the abdomen and neck, dizziness, fainting spells, and chest pain.

In certain aspects of the method, administration of a supportive therapy is also included. Such therapies include, for example, administration of one or more prostacyclins, Endothelin Receptor Antagonists (ERAs) Phosphodiesterase Inhibitors (PDE 5 Inhibitors) Selective IP Receptor Agonists, and Soluble Guanylate Cyclase (sGC) Stimulators Diuretics, Anticoagulants and Oxygen Therapy.

### BRIEF DESCRIPTION OF THE DRAWINGS

FIGS. 1A-1C: Mutagenesis of select uORF start codons mapped by ribosome profiling identifies sequence elements modulating downstream protein-coding ORF expression. FIG. 1A BMPR2 ASO targeting map showing the sequence prior to the BMPR2 CDS start (SEQ ID NO: 37). Blue arrows underneath the map identify sequences encompassed by uORF6 (SEQ ID NO: 35) and uORF9 (SEQ ID NO: 36). Regions targeted by different ASO are also identified. FIG. 1B Schematic representation of BMPR2 5′UTR with all potential uORF s from sorfs.org database (grey) and the subset of uORFs selected for mutagenesis (red) highlighted. FIG. 1C Effect of knocking out each uORF start codon relative to a wild-type BMPR2 5′UTR construct using a dual luciferase assay in HeLa cells. The wild-type start codon for each uORF is displayed above each column. Error bars represent mean+S.D. Data represents the mean of at least 3 biological replicates for each construct tested. Significant changes in luciferase expression are evaluated by two-sided T-test comparing each construct to the negative control. Statistical significance for each comparison is denoted by asterisks (** for P<0.01, * for P<0.05).

FIG. 2: Replication of HeLa luciferase assays in HEK293T cells for selected uORF-disrupting mutations. Effect of knocking out each uORF start codon relative to a wild-type BMPR2 5′UTR construct in dual luciferase assay in HEK293T cells. The wild-type start codon for each uORF is displayed below each column. Error bars represent mean+S.D. Data represents the mean of at least 2 biological replicates for each construct tested. Significant changes in luciferase expression are evaluated by two-sided T-test comparing each construct to the negative control. Statistical significance for each comparison is denoted by asterisks (*** for P<0.001, ** for P<0.01, * for P<0.05).

FIG. 3: qPCR of renilla versus firefly mRNA abundance in HEK293T cells for wild-type versus uORF 6 KO constructs. Error bars represent mean+S.D. Data represents the mean of 3 technical replicates for each construct tested. Significant changes in luciferase expression are evaluated by two-sided T-test comparing each construct to the negative control. Statistical significance for each comparison is denoted by asterisks (*** for P<0.001, ** for P<0.01, * for P<0.05).

FIGS. 4A-4B: Conservation of uORF start codons is associated with effect size of mutations destroying uORF start codons in luciferase experiments. FIG. 4A Mutations affecting uORF start codons that are strongly conserved (phyloP >2) at both the second and third codon positions cause larger changes in luciferase expression compared to those affecting start codons that are not strongly conserved. P-value derived from a two-sided Wilcoxon rank sum test. FIG. 4B PhyloP scores at each base position affected by mutations are correlated with effect size in luciferase experiments. Mutations are labeled by the uORFs they affect. Red dashed line represents the threshold for strongly conserved bases. Grey line represents the simple regression line of magnitude change on phyloP score. P-value derived from Pearson correlation coefficient (r 2=0.49).

FIGS. 5A-5D: Representative immunoblots showing effect of antisense oligonucleotides designed to block uORF translation on BMPR2 protein expression in HeLa cells. FIG. 5A Schematic depicting 18-nt long ASOs designed to block translation initiation at uORF 6 and uORF 9 by targeting sequences proximal to the AUG start site. FIG. 5B Targeting uORF 6, but not uORF 9, increases endogenous BMPR2 protein expression. Western blot for BMPR2 protein expression 24 hours post-transfection with 100 nM ASOs. FIG. 5C Western blot for BMPR2 protein expression examining the effect of varying ASO length on BMPR2 protein expression. FIG. 5D Western blot showing dose-dependence of ASOs targeting uORF 6 for increasing BMPR2 protein expression. Representative immunoblots are shown.

FIGS. 6A-6B: Sequential plasmid and ASO transfection. FIG. 6A HeLa cells were sequentially transfected with the wild-type BMPR2 5′UTR PsiCheck2 plasmid, followed by ASOs targeting uORF 6 and uORF 9. FIG. 6B Renilla/Firefly luciferase expression for each sequential transfection experiment compared to cells transfected with the wild-type BMPR2 5′UTR PsiCheck2 plasmid followed by an empty vehicle. Error bars represent mean+S.D. Data represents the mean of 4 biological replicates for each ASO tested. Significant changes in luciferase expression are evaluated by two-sided T-test comparing relative renilla luciferase expression to cells transfected with uORF 9 targeting ASOs (ASO 9). Statistical significance for each comparison is denoted by asterisks (*** for P<0.001, ** for P<0.01, * for P<0.05).

FIGS. 7A-7C: Representative immunoblots showing effect of ASOs targeting uORF 6 on BMPR2 protein expression in HUVECs. FIG. 7A ASO 62-16 elicits a dose-dependent increase in BMPR2 protein expression after 24 hour transfection in HUVECs. FIG. 7B Increased BMPR2 expression is associated with proportionally increased pSMAD1/5 in HUVECs after 24 hour transfection of ASO 62-16 and 62-LNA. FIG. 7C Treatment of patient-derived pulmonary artery endothelial cells in BMPR2 loss-of-function mutation carriers increases BMPR2 protein expression.

FIG. 8: Plasmid map for BMPR2-PsiCheck2. Plasmid map for PsiCheck2-BMPR2 5′UTR construct. A segment of the BMPR2 5′UTR containing all of the uORFs selected for further analysis was cloned directly upstream of the Renilla luciferase CDS using the NheI and DraIII restriction cut sites as highlighted in brown and green respectively.

FIGS. 9A-9B: FIG. 9A Alignment of the targeted region of the 5′UTR of human (hsa) BMPR2 and mouse (mmu) Bmpr2 gene, along with the ASOs targeting them. The bolded AUG is the translational start of the targeted regulatory uORF. The underlined bases in mASO63 indicate the two sequence changes relative to the human BMPR2-directed ASO63 necessary to ensure complementarity with the mouse ortholog. Note that the ASO sequences are shown as reverse complement to the target (5′ indicated for clarity). FIG. 9B Sequences of mASOctrl and mASO63 as ordered from IDT (m, 2′ O-methyl; *, phosphorothioated).

FIG. 10: WT C57BL/6J mice were injected with ASO at 10 mg/kg of body weight via tail vein. 48 hrs later, the mice were euthanized, and postmortem tissues were collected. Tissue lysates were assayed for murine BMPR2 protein by ELISA. n=8 controls, n=4 uORF-ASO *, p=0.0162 unpaired, two-tailed t-test.

### DETAILED DESCRIPTION

Ribosome profiling, a method that allows for more precise mapping of 5′UTR regulatory elements, has improved 5′UTR annotations and greatly expanded the catalog of uORFs available for therapeutic targeting by ASO technology. By mining publicly available ribosome profiling data, we have identified multiple putative uORFs in the BMPR2 5′UTR sequence that can be modulated to increase downstream protein expression. We have systematically characterized the effect of disrupting translation initiation at a select subset of these uORFs on downstream protein expression in genetic reporter assays. We further describe specific uORF-destroying mutations which can significantly enhance or repress downstream reporter gene expression and have characterized sequence features which correlate with their regulatory effect sizes. Using these data, we designed ASOs specific to the BMPR2 5′UTR that are capable of increasing BMPR2 protein expression by 50-70% in vitro in primary human umbilical vein endothelial cells. Taken together, this work demonstrates the utility of using ribosome profiling to inform target selection for ASO design in 5′UTRs, and identifies novel targeted compounds which increase BMPR2 protein expression with promising translational potential.

Our methodology and specific ASO design provide a targeted approach for increasing BMPR2 expression that can benefit patients with fibrosis generally. However, the compositions described herein are particularly well suited for treating the vascular remodeling and blood vessel dysfunction observed in familial and idiopathic forms of PAH. Because this mechanism of action is at the level of mRNA translation, for patients who have PAH due to loss-of-function mutations in BMPR2 that lead to nonsense-mediated decay of mRNA transcribed from mutant alleles, this treatment strategy will selectively increase protein expression of the intact, wild-type BMPR2 allele.

### Definitions

The present subject matter may be understood more readily by reference to the following detailed description which forms part of this disclosure. It is to be understood that this invention is not limited to the specific products, methods, conditions, or parameters described and/or shown herein, and that the terminology used herein for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. In addition to definitions included in this sub-section, further definitions of terms are interspersed throughout the text.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

As employed above and throughout the disclosure, the following terms and abbreviations, unless otherwise indicated, shall be understood to have the following meanings.

In this invention, “a” or “an” means “at least one” or “one or more,” etc., unless clearly indicated otherwise by context. The term “or” means “and/or” unless stated otherwise. In the case of a multiple-dependent claim, however, use of the term “or” refers back to more than one preceding claim in the alternative only.

A “sample” refers to a sample from a subject that may be tested. The sample may comprise cells, and it may comprise body fluids, such as blood, serum, plasma, cerebral spinal fluid, urine, saliva, tears, pleural fluid, and the like. The sample may also be a tissue sample, or cells derived from a tissue.

As used herein, “subject” includes, but is not limited to vertebrates, more specifically a mammal (e.g., a human, horse, pig, rabbit, dog, sheep, goat, non-human primate, cow, cat, guinea pig or rodent). In preferred embodiments, the subject is a human. The term does not denote a particular age or sex. A patient refers to a subject afflicted with a disease or disorder. The term “patient” includes human and veterinary subjects.

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter, such as a chemical compound, a mixture of chemical compounds, a biological macromolecule, or an extract made from biological materials such as bacteria, plants, fungi, or animal (particularly mammalian) cells or tissues, which, when administered to a subject (human or animal) induces a desired pharmacological and/or physiologic effect by local and/or systemic action.

As used herein the term “wild type” is a term of the art understood by skilled persons and means the typical form of an organism, strain, gene or characteristic as it occurs in nature as distinguished from mutant or variant forms. As used herein the term “variant” should be taken to mean the exhibition of qualities that have a pattern that deviates from the wild type or a comprises non naturally occurring components.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The terms “polynucleotide”, “nucleotide”, “nucleotide sequence”, “nucleic acid”, and “oligonucleotide” are used interchangeably in this disclosure. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: single-, double-, or multi-stranded DNA or RNA, genomic DNA, cDNA, DNA-RNA hybrids, or a polymer comprising purine and pyrimidine bases or other natural, chemically, or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Upstream” and “Downstream” respectively refer to moving along a nucleotide strand in a 3′ to 5′ direction or a 5′ to 3′ direction.

“Introducing into” means uptake or absorption in the cell, as is understood by those skilled in the art. Absorption or uptake of oligos can occur through cellular processes, or via the use of auxiliary agents or devices.

The term “identity” as used herein and as known in the art, is the relationship between two or more oligo sequences, and is determined by comparing the sequences. Identity also means the degree of sequence relatedness between oligo sequences, as determined by the match between strings of such sequences. Identity can be readily calculated (see, e.g., Computation Molecular Biology, Lesk, A. M., eds., Oxford University Press, New York (1998), and Biocomputing: Informatics and Genome Projects, Smith, D. W., ed., Academic Press, New York (1993), both of which are incorporated by reference herein). While a number of methods to measure identity between two polynucleotide sequences are available, the term is well known to skilled artisans (see, e.g., Sequence Analysis in Molecular Biology, von Heinje, G., Academic Press (1987); and Sequence Analysis Primer, Gribskovm, M. and Devereux, J., eds., M. Stockton Press, New York (1991)). Methods commonly employed to determine identity between oligo sequences include, for example, those disclosed in Carillo, H., and Lipman, D., Siam J. Applied Math. (1988) 48:1073. In certain embodiments, the present invention may have 75, 80, 85, 90, 91, 92, 93, 94, 95, 96, 97, 98, 99, or 100% identity with the SEQ ID NOs disclosed herein.

“Substantially identical,” as used herein, means there is a very high degree of homology preferably >90% sequence identity.

The term “exogenous” nucleic acid can refer to a nucleic acid that is not normally or naturally found in or produced by a given bacterium, organism, or cell in nature. The term “endogenous” nucleic acid can refer to a nucleic acid that is normally found in or produced by a given bacterium, organism, or cell in nature.

The term “recombinant” is understood to mean that a particular nucleic acid (DNA or RNA) or protein is the product of various combinations of cloning, restriction, or ligation steps resulting in a construct having a structural coding or non-coding sequence distinguishable from endogenous nucleic acids found in natural systems.

As used herein, “target transcript” means a transcript that encodes a target protein. In certain embodiments, a target transcript contains a primary open reading frame that encodes a primary protein and one or more start sites at which translation of a polypeptide that is not the target protein may be initiated. In certain such embodiments, a target transcript contains a primary open reading frame and a uORF. In certain such embodiments, a target transcript contains a primary open reading frame and more than one uORF. In certain embodiments, a target transcript contains a primary open reading frame and does not contain a uORF. In certain embodiments, a target transcript contains a primary open reading frame and a translation suppression element.

As used herein, “translation suppression element,” means any sequence and/or secondary structure in the 5′-UTR of a target transcript that reduces, inhibits, and/or suppresses translation of the target transcript. In certain embodiments, a translation suppression element comprises a uORF. In other embodiments, a translation suppression element does not comprise a uORF. In certain embodiments, a translation suppression element comprises one or more stem-loops. In certain embodiments, a translation suppression element comprises greater than 60%, greater than 70%, or greater than 80% GC content. In certain embodiments, the translation suppression element is a uORF. In certain embodiments, the translation suppression element is a stem-loop. In certain embodiments, translations suppression of a uORF can increase expression of the pORF.

As used herein, “translation suppression element inhibitor,” means any agent capable of specifically inhibiting the activity of a translation suppression element. In certain embodiments, the activity of a translation suppression element inhibitor is suppression of translation of the pORF polypeptide or protein on the same transcript. For example, translation suppression element inhibitors include nucleic acids (including antisense compounds and siRNA), peptides, antibodies, small molecules, and other agents capable of inhibiting the amount or activity of a translation suppression element.

As used herein, “translation suppression element region” means a portion of the target transcript that comprises one or more translation suppression elements. In certain embodiments, a translation suppression element region comprises a uORF. In certain embodiments, a translation suppression element region comprises more than one uORF. In certain embodiments, a translation suppression element region comprises a uORF and at least one translation suppression element that is not a uORF. In certain embodiments, a translation suppression element region comprises a translation suppression element that is not a uORF and does not contain a uORF.

As used herein, “target protein” means a protein that one desires to increase in amount, concentration, or activity. In certain embodiments, the target protein is encoded by the primary open reading frame of a target transcript.

As used herein, “primary open reading frame” or “pORF” means the portion of the target transcript that encodes the primary protein associated with the transcript. In certain embodiments, the pORF encodes the target protein.

As used herein, “target site” means the portion of the target transcript having a nucleobase sequence that is complementary to a portion of the nucleobase sequence of a modified oligonucleotide. In certain embodiments, the modified oligonucleotide is complementary to the target site across the entire length of the modified oligonucleotide.

As used herein, “start site” means a group of nucleobases on a transcript at which a ribosomal subunit is recruited. In certain embodiments, a start site may result in initiation of translation. In certain embodiments, a start site is an AUG codon. In certain embodiments, a start site is a non-canonical start codon.

As used herein, “upstream open reading frame start site” or “uORF start site” means a start site that is upstream of the pORF start codon. In certain embodiments, a uORF start site initiates translation of a polypeptide that is not the target protein.

As used herein, “uORF” or “upstream open reading frame” means a portion of a target transcript that comprises a start site upstream of (i.e. 5′ of) the pORF and an in frame termination codon. In certain embodiments, a uORF is the portion of the target transcript that is translated when translation is initiated at a uORF start site. In certain embodiments, a uORF does not overlap with a pORF. In certain embodiments, a uORF does overlap with a pORF. In certain embodiments a uORF overlaps with another uORF. In certain embodiments, a uORF is out of frame with a pORF.

As used herein, “uORF polypeptide” means a polypeptide encoded by a uORF. In certain embodiments, a uORF polypeptide is a protein.

As used herein, “uORF inhibitor” means any agent capable of specifically inhibiting the activity of a uORF. In certain embodiments, the activity of a uORF is suppression of translation of the pORF polypeptide or protein on the same transcript. In certain embodiments, the activity of a uORF is suppression of translation of the pORF polypeptide or protein on a different transcript. For example, uORF specific inhibitors include nucleic acids (including antisense compounds and siRNA), peptides, antibodies, small molecules, and other agents capable of inhibiting the amount or activity of a uORF.

“Antisense oligos or strands” are oligos that are complementary to sense oligos, pre-mRNA, RNA or sense strands of particular genes and which bind to such genes and gene products by means of base pairing. When binding to a sense oligo, the antisense oligo need not base pair with every nucleoside in the sense oligo. All that is necessary is that there be sufficient binding to provide for a Tm of greater than or equal to 40° C. under physiologic salt conditions at sub-micromolar oligo concentrations.

“Antisense oligonucleotides” are single stranded oligonucleotides or “oligos” that inhibit the expression of the targeted gene by interfering with some step in the sequence of events leading to gene expression subsequent protein production by directly interfering with the step. Other oligo act by inducing gene target transcript digestion.

“Native RNA” is naturally occurring RNA (i.e., RNA with normal C, G, U and A bases, ribose sugar and phosphodiester linkages).

As used herein, “antisense activity” means any detectable and/or measurable change attributable to the hybridization of an antisense compound to its target nucleic acid.

As used herein, “detecting” or “measuring” means that a test or assay for detecting or measuring is performed. Such detection and/or measuring may result in a value of zero. Thus, if a test for detection or measuring results in a finding of no activity (activity of zero), the step of detecting or measuring the activity has nevertheless been performed.

As used herein, “detectable and/or measurable activity” means a measurable activity that is not zero.

As used herein, “essentially unchanged” means little or no change in a particular parameter, particularly relative to another parameter which changes much more. In certain embodiments, a parameter is essentially unchanged when it changes less than 5%. In certain embodiments, a parameter is essentially unchanged if it changes less than two-fold while another parameter changes at least ten-fold. For example, in certain embodiments, an antisense activity is a change in the amount of a target nucleic acid. In certain such embodiments, the amount of a non-target nucleic acid is essentially unchanged if it changes much less than the target nucleic acid does, but the change need not be zero.

As used herein, “expression” means the process by which a gene ultimately results in a protein. Expression includes, but is not limited to, transcription, post-transcriptional modification (e.g., splicing, polyadenylation, addition of 5′-cap), translation, and post-translational modification.

As used herein, “translation” means the process in which a polypeptide (e.g. a protein) is translated from an mRNA. In certain embodiments, an increase in translation means an increase in the number of polypeptide (e.g. a protein) molecules that are made per copy of mRNA that encodes said polypeptide.

As used herein, “targeting” or “targeted to” means the association of an antisense compound to a particular target nucleic acid molecule or a particular region of a target nucleic acid molecule. An antisense compound targets a target nucleic acid if it is sufficiently complementary to the target nucleic acid to allow hybridization under physiological conditions.

As used herein, “mismatch” means a nucleobase of a first oligomeric compound that is not capable of pairing with a nucleobase at a corresponding position of a second oligomeric compound, when the first and second oligomeric compound are aligned. Either or both of the first and second oligomeric compounds may be oligonucleotides.

The terms “construct”, “cassette”, “expression cassette”, “plasmid”, “vector”, or “expression vector” is understood to mean a recombinant nucleic acid, generally recombinant DNA, which has been generated for the purpose of the expression or propagation of a nucleotide sequence(s) of interest or is to be used in the construction of other recombinant nucleotide sequences.

The term “promoter” or “promoter polynucleotide” is understood to mean a regulatory sequence/element or control sequence/element that is capable of binding/recruiting an RNA polymerase and initiating transcription of sequence downstream or in a 3′ direction from the promoter. A promoter can be, for example, constitutively active, or always on, or inducible in which the promoter is active or inactive in the presence of an external stimulus. Example of promoters include T7 promoters or U6 promoters.

The term “operably linked” can mean the positioning of components in a relationship which permits them to function in their intended manner. For example, a promoter can be linked to a polynucleotide sequence to induce transcription of the polynucleotide sequence.

The terms “complementarity” or “complement” refer to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 4, 5, and 6 out of 6 being 66.67%, 83.33%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 40%, 50%, 60%, 62.5%, 70%, 75%, 80%, 85%, 90%, 95%, 97%, 98%, 99%, or 100%, or percentages in between over a region of 4, 5, 6, 7, and 8 nucleotides, or refers to two nucleic acids that hybridize under stringent conditions.

A nucleic acid as described herein can be “modified” to increase stability in vivo. Such modifications include, without limitation, sugar modifications such as 2′fluoro, 2′-O-methyl, 2′-NH2. The phosphodiester backbone linkage can also be substituted with phosphorothioate as disclosed herein, but other backbone modifications such as triazole linked, or phNA are known to the skilled artisan. Additionally, modified bases can be employed, including without limitation, 7-deaza-dA, and carboxamide-dU. These 2′ substituents influence ASO molecular conformation, resulting in improved RNA target binding affinity and, with the exception of 2′-fluoro, increased nuclease resistance. Like phosphorodiamidate-modified morpholino oligomers, fully 2′-modified ASOs do not recruit RNase Hl. Although less common, nucleobase modifications can also be incorporated into ASO design. Numerous modifications have been identified, e.g., replacing cytosine with 5-methylcytosine has proved beneficial: 5-methylcytosine substitution reduces ASO immunostimulatory effects without compromising Watson-Crick complementarity. Third-generation modifications more extensively alter ASO chemistry to further enhance stability and potency post-administration and provide greater control over both target affinity and cellular tropism. These modifications may alter nucleoside connectivity and restrict ASO stereochemistry, as in locked nucleic acids, constrained ethyl nucleoside analogues and artificial amido-bridged nucleic acids; change the backbone charge (phosphorodiamidate-modified morpholino oligomers are third-generation modified ASOs); or link ASOs to ligands, as in cholesterol- and GalNAc-conjugated ASOs.

In some aspects, the invention provides methods comprising delivering one or more polynucleotides, such as or one or more vectors as described herein (e.g., encoding all or portions of the base editing complexes discussed below), one or more transcripts thereof, and/or one or proteins transcribed therefrom, to a host cell. In some aspects, the invention further provides cells produced by such methods, and organisms (such as animals, plants, or fungi) comprising or produced from such cells. Conventional viral and non-viral based gene transfer methods can be used to introduce nucleic acids in mammalian cells or target tissues. Such methods can be used to administer nucleic acids encoding components of a base editing system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line.

In one aspect, the invention provides for methods of modifying a target polynucleotide in a eukaryotic cell, which may be in vivo, ex vivo or in vitro. In some embodiments, the method comprises sampling a cell or population of cells from a human or non-human animal and modifying the cell or cells. Culturing may occur at any stage ex vivo. The cell or cells may be re-introduced into the human or non-human animal.

### Methods of Treating PAH-Associated Pathophysiology, Including Vasoconstriction Vascular Remodeling and Fibrosis

Encompassed herein are methods of treating PAH-associated pathophysiology in a subject, comprising administering an effective amount of an antisense oligonucleotide. In preferred embodiments, the oligo modulates BMPR2 signaling. By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist”.

The term “inhibit” means to reduce or decrease in activity or expression. This can be a complete inhibition or activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

The term “fibrosis” describes the development of fibrous connective tissue as a reparative response to injury or damage. Fibrosis may refer to the connective tissue deposition that occurs as part of normal healing or to the excess tissue deposition that occurs as a pathological process. When fibrosis occurs in response to injury, the term “scarring” is used. Physiologically, fibrosis acts to deposit connective tissue, which can interfere with or totally inhibit the normal architecture and function of the underlying organ or tissue. Fibrosis can be used to describe the pathological state of excess deposition of fibrous tissue, as well as the process of connective tissue deposition in healing. In certain embodiments encompassed herein, the fibrosis is pulmonary fibrosis, cardiac fibrosis, liver fibrosis. In certain embodiments, the pulmonary fibrosis is pulmonary arterial hypertension.

The term “pulmonary arterial hypertension” or “PAH,” as used herein, refers to a rare, progressive fatal lung disease affecting the cardiovascular system, which is characterized by proliferation and remodeling of vascular endothelial and smooth muscle cells in the small pulmonary arteries and arterioles, resulting in a physical narrowing of the arteries, progressive increases in pulmonary vascular resistance, elevation in pulmonary artery pressure, right heart failure, and eventually, death as well as a broad spectrum of clinical signs and symptoms. PAH may be classified into different disease types, including familial or sporadic PAH. A majority of familial PAH cases result from loss of function frameshift mutations in the BMPR2 gene. Sporadic PAH patients also have reduced BMPR2 expression in pulmonary endothelial cells. BMPR2 mutation carriers who do not develop PAH tend to have higher BMPR2 expression than patients who develop PAH.

The clinical symptoms of pulmonary arterial hypertension may include shortness of breath, fatigue, edema, swelling of the feet, legs and eventually the abdomen and neck, dizziness, fainting spells, and chest pain. Other symptoms include symptoms common to other lung diseases such as asthma, emphysema, or chronic obstructive pulmonary disease (COPD). In early presentations of PAH there may not be any symptoms at all. However, as the disease progresses, symptoms common to other lung diseases, such as those discussed in this paragraph, manifest. A diagnosis of pulmonary arterial hypertension is established on the basis of well characterized clinical signs and symptoms that are well known to skilled persons.

PAH also refers to any other lung disease manifested by the narrowing or blocking of arteries in the lungs. The first symptoms which appear at the onset of PAH may be referred to at times as “PAH-related symptoms.” The signs of PAH in the animal model can include elevated right systolic ventricular pressure, increased mean pulmonary artery pressure, right ventricular hypertrophy, changes in the right ventricle/left ventricle and septum ratio, increased muscularization of the pulmonary artery and associated arterioles, angio-obliterative lesions in the pulmonary arteries, formation of neointimal lesions, and endothelial cell apoptosis. In humans, such first PAH-related symptoms may typically be exertional dyspnea and fatigue, in addition to symptoms associated with right ventricular failure including exertional chest pain, exertional syncope, peripheral edema, anorexia, and abdominal pain and/or swelling, etc.

Clinically, there are 5 subgroups of pulmonary hypertension (PH), and increasing BMPR2 expression is most likely to be beneficial in Group I PAH patients, where the disease is primarily characterized by vascular remodeling due to pulmonary endothelial cell dysfunction, pulmonary endothelial cell death, enhanced pulmonary artery smooth muscle cell proliferation and hypertrophy of the pulmonary arteries and tissue fibrosis. There can be evidence of fibrosis in Group I PAH, but the primary pathology is vascular remodeling and thickening of the pulmonary arteries. Vascular remodeling and fibrosis can also be observed in other clinical subgroups of pulmonary hypertension (Groups II-V) and therefore modulating BMPR2 expression to oppose these processes may also be beneficial in other clinical subgroups of pulmonary hypertension as well.

One of ordinary skill knows that certain medications and procedures can slow the progression of the disease and improve or manage the quality of life of the subject. These include treatment to relax or prevent constriction of the blood vessels and chest, such as prostacyclins, Endothelin Receptor Antagonists (ERAs) Phosphodiesterase Inhibitors (PDE 5 Inhibitors) Selective IP Receptor Agonists, and Soluble Guanylate Cyclase (sGC) Stimulators. Additionally, Diuretics, Anticoagulants and Oxygen Therapy may be used in supportive therapies. In certain embodiments, these medications and procedures may be administered in combination with the ASOs to treat PAH and ameliorate symptoms thereof.

In a preferred embodiment of the invention, a composition for treating PAH by enhancing BMPR2 expression via inhibition of expression of certain uORFs is provided.

### Modes of Administration of Nucleic Acids Reducing PAH Pathophysiology, Including Vasoconstriction, Vascular Remodeling, and Fibrosis Formation in PAH Subjects in Need Thereof.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

For intravenous administration, the compositions are packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates.

The formulation can contain a preservative to prevent the growth of microorganisms. Suitable preservatives include those that will not interfere with the activity of any of the active components of the formulation.

The formulation is typically buffered to a pH of 3-8 for parenteral administration upon reconstitution. Suitable buffers include, but are not limited to, phosphate buffers, acetate buffers, and citrate buffers.

The parenteral formulations described herein can be formulated for controlled release including immediate release, delayed release, extended release, pulsatile release, and combinations thereof.

In certain embodiments the uORF targeting nucleic acids of the invention can be incorporated into microparticles, nanoparticles, or combinations thereof that provide controlled release of the compounds and/or one or more additional active agents. In forms wherein the formulations contain two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, the compounds and/or one or more additional active agents can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug-containing microparticles can be achieved through known pharmaceutical formulation techniques.

To produce a coating layer of cross-linked protein (e.g., antibodies, ligands of receptors of interest) surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

In certain embodiments, (e.g., for the treatment of lung disorders, such as pulmonary fibrosis or to allow for self-administration) it may be desirable to deliver the oligos described herein in aerosolized form. A pharmaceutical composition comprising at least one oligo can be administered as an aerosol formulation which contains the oligos in dissolved, suspended or emulsified form in a propellant or a mixture of solvent and propellant. The aerosolized formulation is then administered through the respiratory system or nasal passages.

An aerosol formulation used for nasal administration is generally an aqueous solution designed to be administered to the nasal passages as drops or sprays. Nasal solutions are generally prepared to be similar to nasal secretions and are generally isotonic and slightly buffered to maintain a pH of about 5.5 to about 6.5, although pH values outside of this range can also be used. Antimicrobial agents or preservatives can also be included in the formulation.

An aerosol formulation for use in inhalations and inhalants is designed so that the oligos are carried into the respiratory tree of the patient. See (WO 01/82868; WO 01/82873; WO 01/82980; WO 02/05730; WO 02/05785. Inhalation solutions can be administered, for example, by a nebulizer. Inhalations or insufflations, comprising finely powdered or liquid drugs, are delivered to the respiratory system as a pharmaceutical aerosol of a solution or suspension of the drug in a propellant.

An aerosol formulation generally contains a propellant to aid in disbursement of the oligos. Propellants can be liquefied gases, including halocarbons, for example, fluorocarbons such as fluorinated chlorinated hydrocarbons, hydrochlorofluorocarbons, and hydrochlorocarbons as well as hydrocarbons and hydrocarbon ethers (Remington's Pharmaceutical Sciences 18th ed., Gennaro, A. R., ed., Mack Publishing Company, Easton, Pa. (1990)).

Halocarbon propellants useful in the invention include fluorocarbon propellants in which all hydrogens are replaced with fluorine, hydrogen-containing fluorocarbon propellants, and hydrogen-containing chlorofluorocarbon propellants. Halocarbon propellants are described in Johnson, U.S. Pat. No. 5,376,359, and Purewal et al., U.S. Pat. No. 5,776,434.

Hydrocarbon propellants useful in the invention include, for example, propane, isobutane, n-butane, pentane, isopentane and neopentane. A blend of hydrocarbons can also be used as a propellant. Ether propellants include, for example, dimethyl ether as well as numerous other ethers.

The oligos can also be dispensed with a compressed gas. The compressed gas is generally an inert gas such as carbon dioxide, nitrous oxide or nitrogen.

An aerosol formulation of the invention can also contain more than one propellant. For example, the aerosol formulation can contain more than one propellant from the same class such as two or more fluorocarbons. An aerosol formulation can also contain more than one propellant from different classes. An aerosol formulation can contain any combination of two or more propellants from different classes, for example, a fluorohydrocarbon and a hydrocarbon.

Effective aerosol formulations can also include other components, for example, ethanol, isopropanol, propylene glycol, as well as surfactants or other components such as oils and detergents (Remington's Pharmaceutical Sciences, 1990; Purewal et al., U.S. Pat. No. 5,776,434). These aerosol components can serve to stabilize the formulation and lubricate valve components.

The aerosol formulation can be packaged under pressure and can be formulated as an aerosol using solutions, suspensions, emulsions, powders and semisolid preparations. A solution aerosol consists of a solution of an active ingredient such as oligos in pure propellant or as a mixture of propellant and solvent. The solvent is used to dissolve the active ingredient and/or retard the evaporation of the propellant. Solvents useful in the invention include, for example, water, ethanol and glycols. A solution aerosol contains the active ingredient peptide and a propellant and can include any combination of solvents and preservatives or antioxidants.

An aerosol formulation can also be a dispersion or suspension. A suspension aerosol formulation will generally contain a suspension of an effective amount of the oligos and a dispersing agent. Dispersing agents useful in the invention include, for example, sorbitan trioleate, oleyl alcohol, oleic acid, lecithin, and corn oil. A suspension aerosol formulation can also include lubricants and other aerosol components.

An aerosol formulation can similarly be formulated as an emulsion. An emulsion can include, for example, an alcohol such as ethanol, a surfactant, water and propellant, as well as the active ingredient, the oligos. The surfactant can be nonionic, anionic or cationic. One example of an emulsion can include, for example, ethanol, surfactant, water and propellant. Another example of an emulsion can include, for example, vegetable oil, glyceryl monostearate and propane.

The following materials and methods are provided to facilitate the practice of the present invention.

Identification of Putative uORFs in BMPR2 5′UTR

All ORFs (n=30) from the sorfs.org database between the genomic coordinates chr2:202,376,436-chr2:202,377,551 were downloaded and filtered to select only putative uORFs starting with cognate, or near-cognate (AUG, CUG, GUG, UUG) start codons (n=25). From this set of putative uORFs, 10 uORFs were selected for generating luciferase expression vectors. Putative uORFs using cognate AUG start codons, uORFs closer to the BMPR2 CDS, were prioritized. We also sought to maximize coverage of different clusters of uORFs which shared the same stop codon across the annotated BMPR2 5′UTR (ENST00000374580).

### Construction of Expression Vectors

We selected an 860 BP fragment of the BMPR2 5′UTR sequence (1145 bp total) to clone upstream of the Renilla luciferase coding sequence in the PsiCheck-2 plasmid backbone (Promega Cat. #C8021). The BMPR2 5′UTR fragment excludes the first 288 bases of the ENST00000374580 BMPR2 transcript 5′UTR annotation, which contained several complex GC-rich regions. The remaining 860 BP fragment encompasses all of the uORFs selected for mutagenesis studies and alternative shorter (539, 387 bp) BMPR2 5′UTR sequences that have been previously reported in the literature [25,26]. This fragment was inserted upstream of the Renilla luciferase ORF using the NheI and DraIII restriction cut sites on the plasmid backbone. For cloning, we added the first 200 base pairs of the Renilla luciferase ORF adjacent to the 3′ end of the BMPR2 5′UTR fragment to replicate the exact relationship between the BMPR2 5′UTR sequence and BMPR2 coding sequence start. The correct insert was verified by Sanger sequencing of both the positive and negative strands using primers upstream and downstream of the ligation site on the PsiCheck-2 backbone.

BMPR2 5′UTR mutant plasmids were generated by site-directed mutagenesis (New England BioLabs Cat. #E0554S). Primers for site-directed mutagenesis were designed using the NEBaseChanger (version 1.3.0) online tool (https://nebasechanger.neb.com/). For all constructs, the desired mutation was confirmed by Sanger sequencing in both forward and reverse directions.

### Cell Culture and Transfections

HeLa or HEK293T cells were used for conditional expression of reporter genes. For transient transfections, cells were split 1 day before transfection and seeded in 6-well plates at a density of 400,000 cells per well. 0.2 μg of the BMPR2-PsiCheck-2 dual luciferase plasmid was transfected into each well using Lipofectamine 3000 following the manufacturer's protocol. Biological replicates were obtained by transfecting cells from separate passages on separate days using newly prepared reagents. HeLa and HEK293T cells were cultured in Dulbecco's modified Eagle's medium (DMEM) supplemented with 10% (v/v) fetal bovine serum and antibiotics. For plasmid-ASO sequential transfection experiments, HeLa cells were seeded in 10 cm dishes and transfected at 70-80% confluence with 0.6 μg of the wild-type BMPR2-PsiCheck-2 dual luciferase plasmid. 16-hours post-transfection, cells were trypsinized, re-seeded into 6 well plates, and re-transfected 8 hours later with antisense oligonucleotides using lipofectamine RNAiMax following the manufacturer's recommended protocol. ASO-transfected cells were incubated for 16 hours prior to harvest.

Primary human umbilical vein endothelial cells (HUVECs) passage 3-6 were cultured using Lonza EGM-2 complete growth medium (Lonza Cat. #CC-3162) in 10 cm dishes pre-treated with EmbryoMax 0.1% Gelatin Solution (Millipore Cat. #ES-006). HUVECs were transfected at 80-90% confluence using 1.2 μL/mL concentration of RNAiMax after preparing ASO-transfection complexes at the indicated concentrations.

### Luminometry Assays

Luminescence was measured using the Promega Dual-Luciferase Reporter Assay System (E1910) following the manufacturer's protocol. Cells were lysed by adding 100 μL of lysis buffer 10 μL of each lysate was transferred to a black opaque 96-well plate. The ratio of Firefly to Renilla luminescence with a microplate reader by automatic injection of the Luciferase Assay Reagent II and Stop & Glo reagents. Biological replicates were obtained by transfecting cells from separate passages on separate days using newly prepared reagents. Luminescence measurements for mutated constructs were normalized using the wild-type construct, and compared across transfections. Statistical significance was determined using a two-sided T-test comparing relative renilla to firefly expression ratio of each test construct normalized to the wild-type construct.

Quantification of mRNA

Quantitative polymerase chain reaction (qPCR) was performed to quantify relative firefly versus renilla mRNA in transfected HeLa cells using SYBR Green. RNA was extracted from HeLa cells 24 hours post-transfection with indicated plasmid constructs using Trizol. The following primers were used: Firefly Forward: GACACCGCTATTCTGAGCGT (SEQ ID NO: 1), Firefly Reverse: CTCGAAGCGGTACATCAGCA (SEQ ID NO: 2), Renilla Forward: CACTGTCTAAGGTGGGC (SEQ ID NO: 3), Renilla Reverse: GGGGTAATCAGAATGGCGCT (SEQ ID NO: 4). Primers were synthesized by Integrated DNA Technologies.

### Semi-Quantitative Immunoblotting

Cells were harvested for immunoblotting 16-24 hours post-transfection with ASO-RNAiMax complexes. Cells were washed 2× with ice-cold PBS, detached by mechanical scraping, and spun down at 500×g in a table-top centrifuge. The resultant cell pellets were lysed by 30 minute incubation on ice in RIPA buffer supplemented with 1×PMSF and cOmplete protease inhibitor. For phospho-protein blotting experiments, the phosphatase inhibitors sodium orthovandate and sodium fluoride were added to the lysis buffer. Lysate protein content was determined by BCA assay following the manufacturer's protocol. 20-40 ug of cell lysates were loaded for each sample for SDS-PAGE and relative protein abundances were determined using Image Studio Lite (version 5.2) by dividing the median intensity signal for each protein of interest against an internal loading control (GAPDH, β-Actin, or Ku70 for all experiments). Statistical significance was determined by a one-sample T-test using normalized intensity values for each protein across at least 3 independent biological replicates.

The following primary antibodies with associated catalogue numbers were used for all immunoblotting experiments: BMPR2 (3F6) mouse mAb (Millipore Sigma Cat. #MABD171); GAPDH (14C10) Rabbit mAb (Cell Signaling Technology Cat #. 2118); Phospho-Smad1/5 (Ser463/465) (41D10) Rabbit mAb (Cell Signaling Technology Cat #. 9516); β-Actin (8H10D10) Mouse mAb (Cell Signaling Technology Cat #. 3700); Ku70 (D10A7) Rabbit mAb (Cell Signaling Technology Cat #. 4588).

The following secondary antibodies were used for all immunoblotting experiments: IRDye 680RD Donkey anti-Rabbit IgG secondary (LICOR 925-68073); IRDye 800CW Donkey anti-Mouse iGG secondary (LICOR 925-32212).

The following examples are provided to illustrate certain embodiments of the invention. They are not intended to limit the invention in any way.

### Example 1: Mapping Cis-Regulatory Translational Elements in the BMPR2 5′UTR

Our search of ORFs mapped to the 5′UTR sequence of the primary BMPR2 transcript (ENST00000374580) uncovered 30 putative uORFs identified by the sorf. org ORF-mapping pipeline. To identify which uORF candidates are most repressive on downstream BMPR2 protein-coding ORF (pORF) expression, we first filtered this set of uORFs to keep only those using near-cognate (CUG, GUG, UUG) or cognate (AUG) start codons as these sequences have previously been shown to be most capable of facilitating ribosome translation initiation [11], leaving us with 25 possible uORFs. When we mapped each of these uORFs to the full-length BMPR2 5′UTR sequence, we observed that many putative uORFs shared the same stop codon, possibly reflecting the limited capacity for ribosome profiling to precisely determine sites of translation initiation or that some uORFs could initiate translation at multiple upstream start sites.

From this set of 25 possible uORFs in the 5′UTR of BMPR2 we selected 10 uORFs for mutagenesis, prioritizing uORFs using cognate AUG start codons, uORFs closer to the BMPR2 CDS, and finally maximizing coverage of different clusters of uORFs which shared the same stop codon across the BMPR2 5′UTR (see Table 1 for details of selected uORFs).

TABLE 1
Features Associated with uOFRs mapped in the BMPR2 5′ UTR

| ORF | | Genomic | Genomic | Start | ORF |
|---|---|---|---|---|---|
| number | Chrom | Start | End | codon | length (bp) |
| 1 | 2 | 202376619 | 202376660 | TTG | 42 |
| 2 | 2 | 202376965 | 202376997 | CTG | 33 |
| 3 | 2 | 202376988 | 202377056 | ATG | 69 |
| 4 | 2 | 202377000 | 202377056 | CTG | 57 |
| 5 | 2 | 202377106 | 202377168 | CTG | 63 |
| 6 | 2 | 202377112 | 202377168 | ATG | 57 |
| 7 | 2 | 202377170 | 202377307 | TTG | 138 |
| 8 | 2 | 202377185 | 202377307 | TTG | 123 |
| 9 | 2 | 202377210 | 202377284 | ATG | 75 |
| 10 | 2 | 202377221 | 202377307 | TTG | 87 |

Next, to interrogate the functionality of each uORF mapped by ribosome profiling, we cloned a fragment of the BMPR2 5′UTR containing all of the selected uORFs upstream of the luciferase protein-coding ORF in a dual-luciferase plasmid backbone. We systematically generated mutant constructs for each of the selected uORFs by introducing a single point mutation changing each NUG uORF start codon to NAG by site-directed mutagenesis (Table 2).

TABLE 2
Primers used for site-directed mutagenesis

| uORF | Mutation | Forward* | Reverse** | Ta |
|---|---|---|---|---|
| 1 | TTG > TAG | CTAGCTTTCTaGGTGGAATTTAC | CCTATAGTGAGTCGTATTAAC | 59 |
| 2 | ATG > AAG | AGCTTGTCCAaGGAGGCAGGC | CACAGTCCCCACGCCTTC | 70 |
| 3 | CTG > CAG | TAAGGAATCCaGCCTTCCCGG | AGTTTCGCTTCGGTGCTT | 65 |
| 4 | CTG > CAG | CCGCGGGCGAaGCGACTAGGG | CTCCGGGAAGGCAGGATTCCTTAAG | 72 |
| 5 | CTG > CAG | CTCCGCACCCaGGATATGTTTTCTCC | GGCGGGGATTCGATCCGG | 69 |
| 6 | ATG > AAG | ACCCTGGATAaGTTTTCTCCCAGACCTGG | GCGGAGGGCGGGGATTCG | 72 |
| 7 | TTG > TAG | GGAAATAATTaGGGGGATTTCTTC | CTCGTAGTTTCACGATATC | 58 |
| 8 | TTG > TAG | GATTTCTTCTaGGCTCCCTGC | CCCCAAATTATTTCCCTC | 58 |
| 9 | ATG > AAG | CCCACAGACAaGCCTTCCGTT | GAAAGCAGGGAGCCAAGAAG | 67 |
| 10 | TTG > TAG | GCCTTCCGTTaGGAGGGCCGC | ATGTCTGTGGGGAAAGCAGGG | 70 |
| Neg. | CTG > CAG | GAACTAGTTCaGACCCTCGCCCC | CCGACTGCCGGGAAGGAC | 69 |
| CTRL | | | | |

*SEQ ID Nos. 5-15 are shown in descending order.
**SEQ ID Nos. 16-26 are shown in descending order.

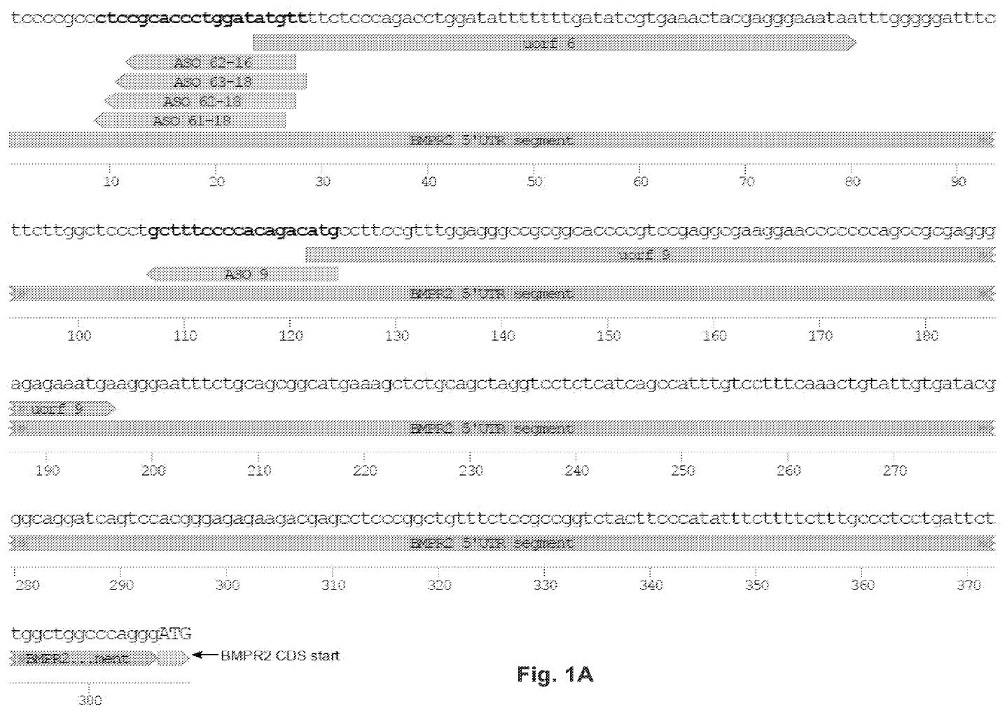

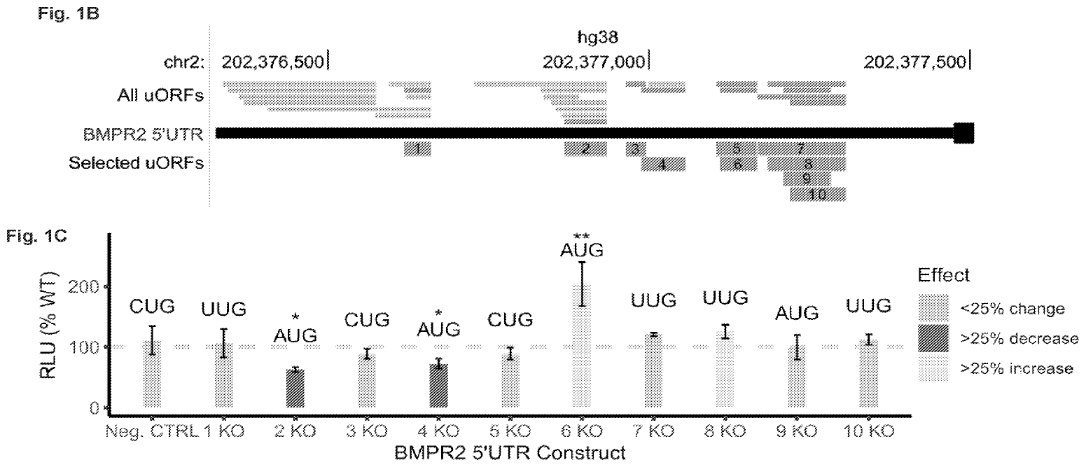

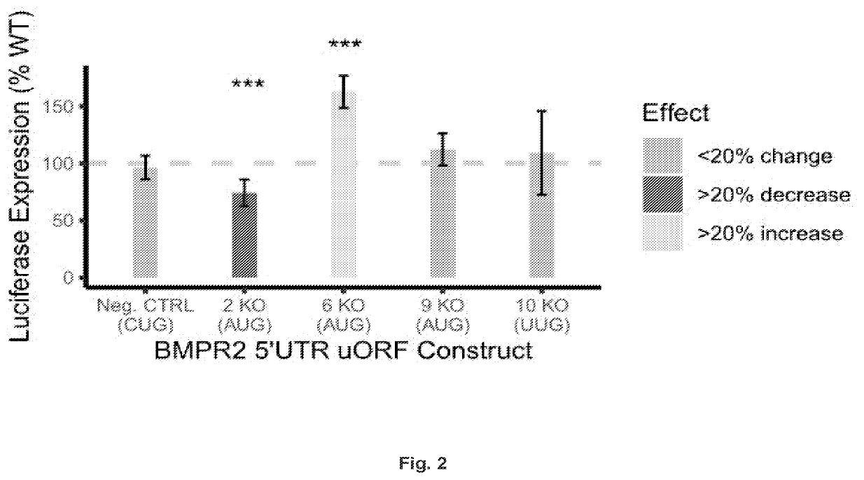

As a negative control, we generated an additional construct with a CUG>CAG point mutation outside of an annotated uORF start codon. We then compared the effect of each uORF-destroying point mutation through dual luciferase assay in HeLa and HEK293T cell lines (FIG. 1b, FIG. 2).

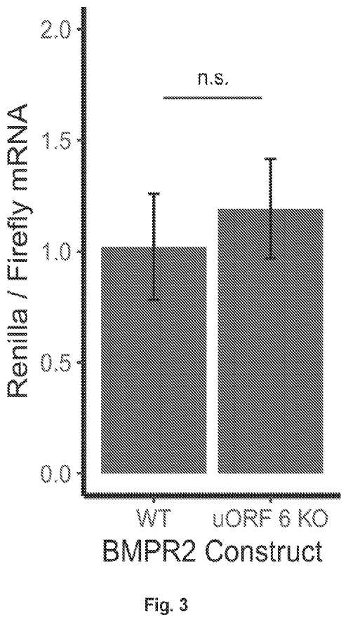

Although mutagenesis produced mostly modest effects on luciferase expression, we found that knock-out of uORF 6 caused a striking 1.5-2 fold increase in luciferase signal compared to the wild-type construct. The specific effect of uORF 6 KO on luciferase translation was confirmed by qPCR which showed no difference in Renilla luciferase mRNA levels after transfection (FIG. 3). Surprisingly, mutagenesis of uORF 2 and 4 start codons each decreased luciferase expression, suggesting that translation of these uORFs could enhance translation at the protein-coding ORF. The observed pattern of decreasing luciferase expression with KO of uORFs 2 and 4 and increasing luciferase expression with KO of uORF 6 is consistent with the possibility that translation initiation at uORFs upstream of uORF 6 may release the BMPR2 pORF from translational repression by uORF 6.

Mutating start codons from other AUG-initiated uORFs in the BMPR2 5′UTR has tended to cause pronounced changes in luciferase expression. In general, the stronger effect size of mutating AUG start codons compared to near-cognate start codons is consistent with previous observations that cognate AUG start codons initiate translation much more efficiently [11].

We next asked whether effect sizes from our luciferase experiments could be modeled using features in the ribosome profiling data. For this analysis we selected the subset of ribosome profiling statistics associated with a single study which had mapped all of the uORFs in our mutagenesis experiments [12]. To remove non-independent observations from the data we reduced multiple uORFs sharing the same reading frame and stop codon to a single observation by selecting the uORF with the greatest per Kilobase per Million mapped reads (RPKM) in each set. We then modeled the absolute change in luciferase expression with each uORF-KO construct as a function of each uORF's RPKM, tendency for ribosome-protected fragments to accumulate in the mapped ORF reading frame (Orfscore), start codon used, and ORF length using multivariable linear regression. The resultant model was not significant (Adjusted r 2=0.54, p=0.4502), suggesting that uORF sequence features in combination with statistics from ribosome profiling experiments poorly predict the magnitude of the effect of uORF start codon KO on downstream protein expression in linear models.

### Example 2: Conservation of the uORF Start Codon is Associated with Larger Effect Sizes

To further explore possible features correlating with effects observed in luciferase experiments, we asked whether there was correlation between the degree that uORF start codons are evolutionarily conserved and their downstream effect on luciferase expression. Here we examined phyloP scores for the second and third uORF start codon positions, where mutations affecting annotated uORF start codons cannot result in cognate or near-cognate start codon. A uORF start codon was considered to be strongly conserved if both its second and third nucleotide positions had phyloP scores >2, corresponding to strong conservation of the base [13]. When we divide uORFs into those with or without strongly conserved start codons, we observe that strongly conserved uORF start codons produce significantly greater effects on luciferase expression when mutagenized compared to weakly conserved start codons (FIG. 4a, Wilcoxon Rank Sum P=0.017). Additionally, we further observed that the phyloP score of the mutated start codon base was capable of accounting for −49% of the variation in changes in luciferase expression in our experiments using simple linear regression (FIG. 4b, r 2=0.49, P=0.024), demonstrating that mutations affecting more strongly conserved bases in uORF start codons may have greater effects on downstream protein expression. More broadly, these associations between uORF start codon conservation and magnitude of the effect on luciferase expression is consistent with previous findings that more strongly conserved uORF initiation codons are also more strongly inhibitory [14].

### Example 3: Antisense Oligonucleotides Blocking BMPR2 uORFs Increase Protein Expression in HeLa Cells

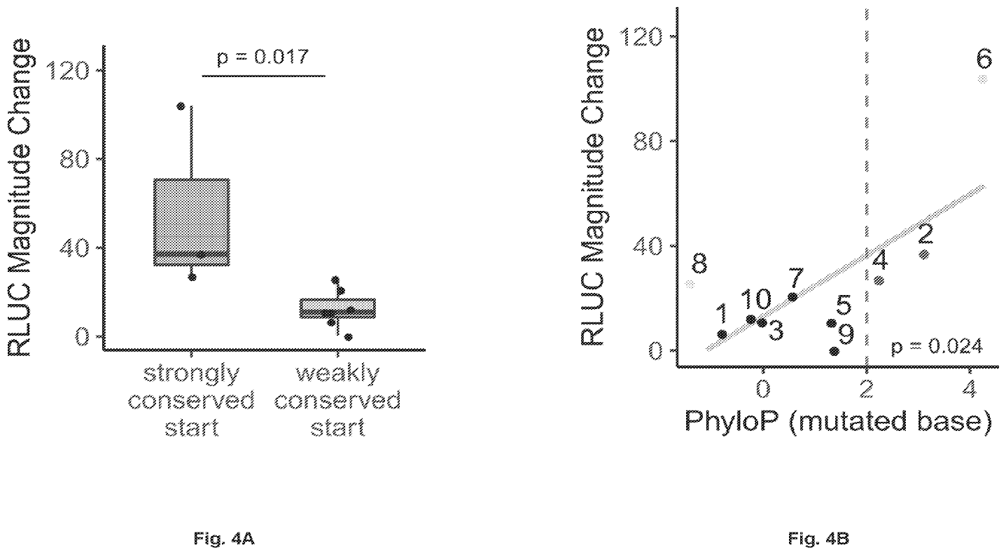

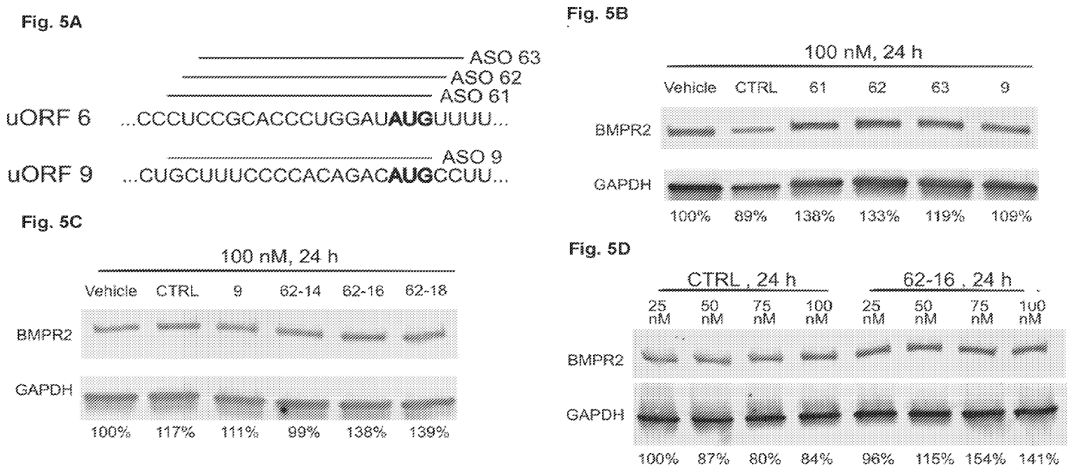

Given results from our luciferase experiments, we designed a panel of 18-nucleotide antisense oligonucleotides (ASOs) with base complementarity to the start codon and immediate upstream region of uORF 6 to identify ASOs capable of increasing BMPR2 production by blocking inhibitory elements in the 5′UTR (FIG. 5a). As negative controls, we designed a scramble ASO sequence with no homology to annotated human mRNA sequences (CTRL), and used an additional 18-nucleotide ASO targeting uORF 9, which was shown to have a minimal effect on downstream protein expression by luciferase assay.

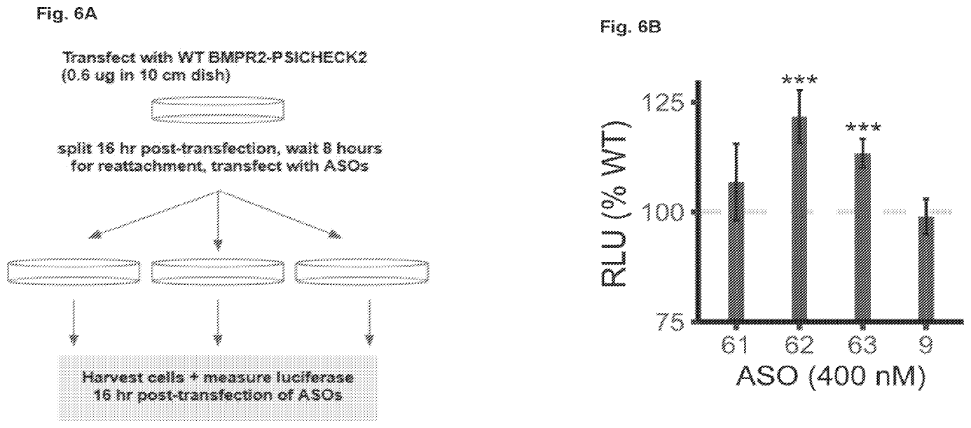

To test the ability of these ASOs to increase BMPR2 protein expression, we co-transfected the BMPR2 wild-type 5′UTR luciferase construct followed by each uORF-targeting ASO (FIG. 6). Results from these experiments showed that co-transfection with uORF 62 and 63 produced an increase in luciferase expression, while as predicted, co-transfection with an ASO targeting uORF 9 had no effect. To confirm that ASOs targeting uORF 6 could increase BMPR2 protein expression, we transfected each ASO into HeLa cells and assessed endogenous BMPR2 protein levels by western blot 24-hours post transfection. Consistent with previous results from the luciferase mutagenesis and luciferase co-transfection experiments, we observed that ASOs targeting uORF 6 were capable of increasing endogenous BMPR2 protein expression in HeLa cells, while an ASO targeting uORF 9 had no effect (FIG. 5b). Further supporting the ability for ASOs targeting uORF 6 to increase endogenous BMPR2 protein expression, we observed that the potency of ASOs targeting uORF 6 were length-dependent (FIG. 5c), and dose-dependent (FIG. 5d).

TABLE 3
ASO sequences used

| | | | SEQ |
|---|---|---|---|
| Name | Sequence | BMPR2 Activity | ID NO: |
| CTRL | CAUGGAGGCGGCUUGAG | − | 27 |
| 61-18 | CAUAUCCAGGGUGCGGAG | + | 28 |
| 62-14 | ACAUAUCCAGGGUG | − | 29 |
| 62-16 | ACAUAUCCAGGGUGCG | + | 30 |
| 62-18 | ACAUAUCCAGGGUGCGGA | + | 31 |
| 63 | AACAUAUCCAGGGUGCGG | + | 32 |
| mASO63 | AACAUAUCCAGAGUGCCG | + in vivo in | 38 |
| | | mouse pulmonary | |
| | | artery | |
| 62-LNA** | ACAUAUCCAGGGU + G + | + | 30 |
| | C + G | | |
| 9 | CAUGUCUGUGGGGAAAGC | − | 33 |
| 62-mouse | ACAUAUCCAGAGUGCC | + (in mouse | 34 |
| | | cells) | |

*All ASOs have completely 2′O-methyl modified bases and
phosphorothioate backbones unless otherwise noted
**This is the same sequence as “62-16” but the last 3
nucleotides are modified with LNA chemistry (+), all
other bases are methyl modified and all bases are
connected by a phosphothiorate backbone

### Example 4: Antisense Oligonucleotides Blocking Translation of uORF 6 Increase BMPR2 Protein Expression in HUVECs

Given our initial results in HeLa cells, we sought to demonstrate the generalizability of our approach by determining whether ASOs targeting uORF 6 could produce a similar increase in BMPR2 protein expression in primary human cell lines. We selected human umbilical vein endothelial cells given their availability and the centrality of endothelial cell dysfunction in the pathophysiology of PAH.

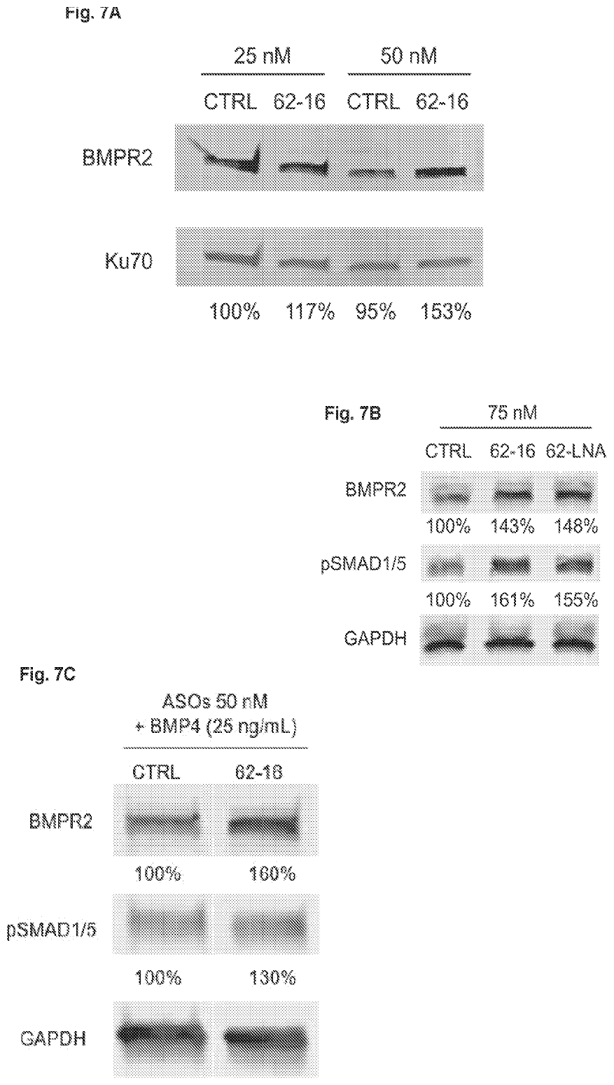

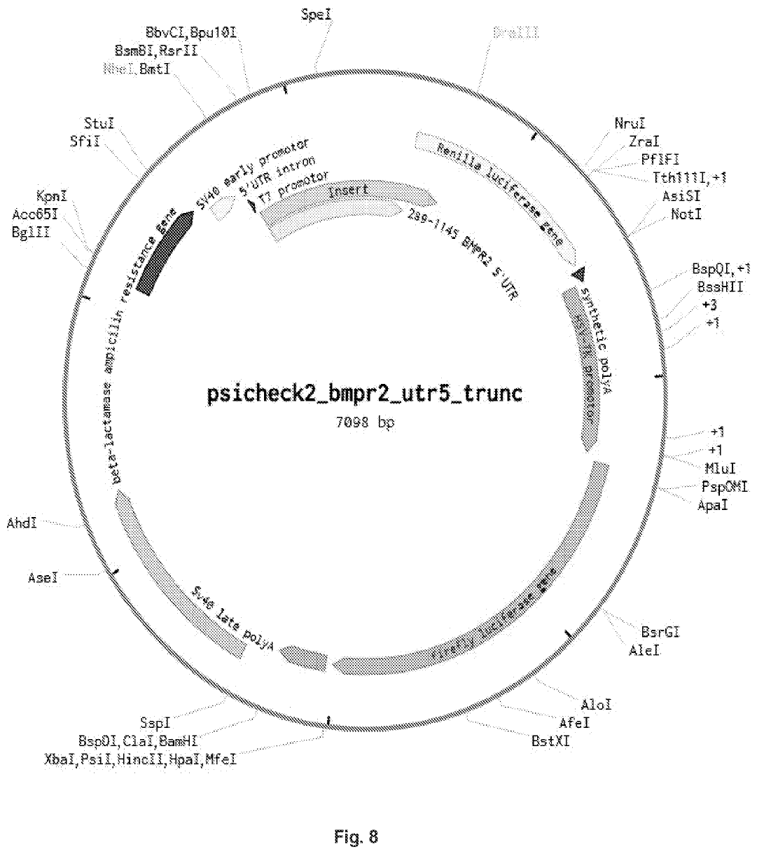

Upon transfection of ASOs targeting uORF 6 in HUVECs we observed a similar increase in BMPR2 protein expression (FIG. 7a), confirming that the effects of blocking uORF 6 observed in HeLa cells could be replicated in a more physiologically relevant cell line. To ascertain whether this increase in BMPR2 protein was functional we measured phosphorylation of the downstream signaling protein SMAD1/5 which is known to become activated upon BMPR2 receptor activation. Here too, we observe that 24 hours post-transfection, treatment with BMP4 ligand induces enhanced phosphorylation of SMAD1/5 in ASO 62 transfected compared to negative-control transfected HUVECs, consistent with an increase in signaling capacity of the BMPR2 receptor complexes (FIG. 4b). A similar result was observed when using a modified ASO (62-LNA) containing locked nucleic acids.

Our experiments have also identified specific ASOs targeting 5′UTR uORF of BMPR2 which initiates translation 363 base pairs upstream of the annotated BMPR2 CDS in the BMPR2 mRNA transcript. These sequences successfully targeted a synthetic BMPR2 5′UTR sequence to increase gene expression in a reporter assay and demonstrated increases in BMPR2 protein with western blot after ASO treatment. These ASO each have 2-O′Methyl modified bases and are connected by phosphorothioate bonds, however other modifications are known in the art (e.g., LNA as described herein above) and can also be included to enhance stability and/or efficacy. These four ASOs are identified in Table 3 above, SEQ ID NOS: 28, 31, 30 and 32.

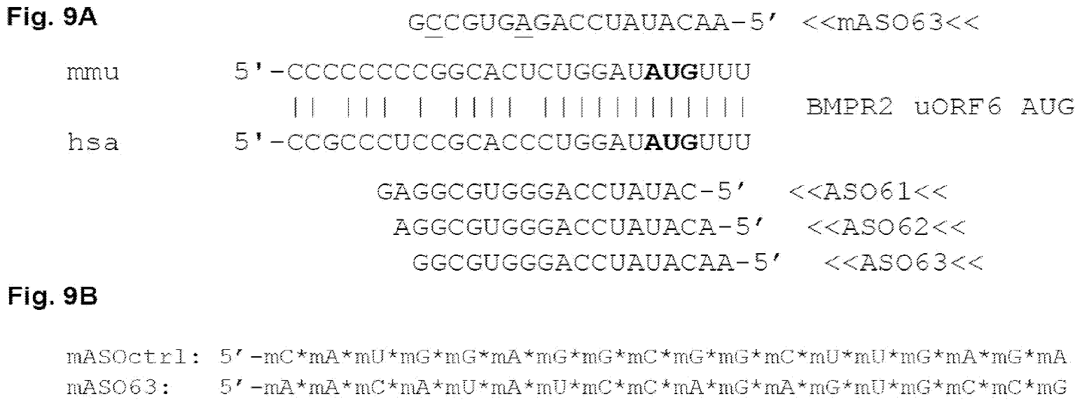

Alignment of the targeted region of the 5′UTR of human (hsa) BMPR2 and mouse (mmu) Bmpr2 gene, along with the ASOs targeting them is shown in FIG. 9A. The bolded AUG is the translational start of the targeted regulatory uORF. The underlined bases in mASO63 indicate the two sequence changes relative to the human BMPR2-directed ASO63 necessary to ensure complementarity with the mouse ortholog. Note that the ASO sequences are shown as reverse complement to the target (5′ indicated for clarity). FIG. 9B shows the modified sequences of mASOctrl and mASO63 (m, 2′ O-methyl; *, phosphorothioated).

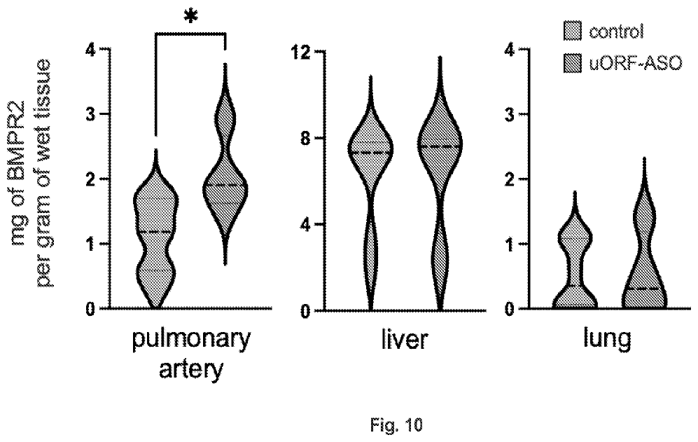

Adult male C57BL/6J mice were anesthetized using inhaled isoflurane, and injected with a single bolus of antisense oligonucleotide at 10 mg per kg of body weight, either targeting the murine Bmpr2 5′UTR (mASO63, see FIG. 9), a scrambled-sequence non-targeting ASO (mASOctrl), or vehicle (phosphate-buffered saline) alone. Both ASO had fully phosphorothioated backbones and contained 2′-O-Me modifications at every nucleotide (Integrated DNA Technologies, Coralville, IA). 48 hours after treatment, the animals were euthanized and pulmonary artery, liver, and lung were harvested, and flash-frozen in liquid nitrogen. Tissue homogenates were prepared by grinding the frozen tissue in PBS containing protease inhibitors (cOmplete™, Mini, EDTA-free Protease Inhibitor Cocktail; Sigma-Aldrich). Levels of murine Bmpr2 protein were quantified by ELISA (Novus Biologicals, Centennial, CO; Part number NBP2-70008) according to the manufacturer's instructions, and the mass of tissue expressed was as a ratio to the wet tissue weight of the sample from which it was derived. FIG. 10 shows the mg of BMPR2 per gram of wet tissue in control and ASO treated mice. Murine BMPR2 protein was assayed by ELISA. n=8 controls, n=4 uORF-ASO *, p=0.0162 unpaired, two-tailed t-test.

### Discussion

We used ribosome profiling data from publicly available resources to map functional uORFs in the 5′UTR of BMPR2 and confirmed their ability to regulate downstream pORF expression with reporter gene assays. Based on these regulatory mapping experiments, we designed ASOs to therapeutically modulate endogenous BMPR2 protein expression by targeting a single uORF in the 5′UTR of BMPR2 found to significantly repress downstream pORF expression. We have shown across multiple cell lines that treatment with ASOs targeting uORF 6 induces an increase in BMPR2 protein production. Given the central role of BMPR2 loss-of-function in the pathogenesis of familial and idiopathic PAH, the identified ASOs represent promising compounds for further therapeutic development. Our findings have several implications for future attempts to prioritize suitable 5′UTR uORF targets for ASO-modulation using ribosome profiling. Given a set of multiple putative uORFs mapped to a gene's 5′UTR sequence, priority should be given to uORFs with cognate start codons, uORFs with strongly conserved start codons, and finally uORFs with shorter intercistronic distances to the pORF. From our study of BMPR2, uORF 6 fulfills all of the above criteria.

Complex cis-regulatory relationships mediated by multiple 5′UTRs uORFs have previously been observed across a number of different genes [20-23]. Results from our luciferase experiments suggest that BMPR2 is similarly subject to multiple layers of translational control. Curiously, we found that knocking out translation initiation in uORFs 2 and 4 decreased pORF expression in luciferase assays. The spatial relationship between uORFs 2 and 4 suggest that earlier translation at these uORFs may help relieve the BMPR2 pORF from translational repression by uORF 6. Alternatively, it is possible that translation at these uORFs resolves secondary structures which could hinder pORF translation initiation. Additionally, our analysis of the ribosome profiling data also uncovered several overlapping uORFs sharing the same stop codon. Mutagenesis of a cluster of these uORFs (uORF 7, 8, and 10) near the luciferase pORF had moderate effects on pORF expression, suggesting that translation in each of these uORFs only repress downstream coding sequences. It remains possible that these clusters of uORFs could have additive effects by presenting the ribosome multiple opportunities to initiate translation with multiple near-cognate start codons sharing the same reading frame.

Among the strategies that have been proposed to increase BMPR2 activity in PAH include using antisense oligonucleotides (ASOs) to sequester micro-RNAs implicated in controlling BMPR2 mRNA levels, compounds capable of inducing translational readthrough of stop codons, and targeting various components of the protein degradation and autophagy pathways. While these approaches are effective at increasing BMPR2 protein levels in vitro, the lack of target specificity limits their translational potential. More targeted approaches have included using viral vectors to mediate BMPR2 gene delivery to the pulmonary endothelium and have been shown to reduce histological hallmarks of PAH, including arterial wall thickening and fibrosis in the pulmonary artery [24]. Like gene delivery approaches, use of ASOs targeting the BMPR2 5′UTR affords more specific target modulation than other less-targeted approaches, with the additional advantage of avoiding the use of viral vectors.

The majority of PAH patients with BMPR2 loss-of-function mutations are thought to carry frameshift mutations which introduce premature termination codons and subject the mutated BMPR2 mRNA transcript to nonsense-mediated decay. A subset of PAH patients with BMPR2 loss-of-function harbor mutations which allow the encoded BMPR2 mRNA to escape nonsense mediated decay and produce a protein product which is capable of reducing BMPR2 signaling by a dominant-negative mechanism. Given the lack of specificity for uORF-targeting ASOs to discriminate between the mutant and wild-type BMPR2 transcript, strategies to recover BMPR2 protein expression by blocking 5′UTR regulatory elements are likely beneficial in the subset of patients in which BMPR2 loss-of-function arises as a consequence of nonsense-mediated decay rather than through a dominant-negative mechanism.

In two separate cell lines, we have identified specific uORF-destroying mutations which lead to 1.5-2.5 fold increases in reporter gene expression. One affects a uORF which begins 363 base pairs upstream of the BMPR2 coding sequence (CDS)—termed “uORF 6”. The second affects a uORF that begins 265 nucleotides upstream of the BMPR2 CDS—termed “uORF 9”.

Crucially, the increase in protein expression associated with mutation of the uORF 9 start codon requires simultaneously disrupting 1-3 base pairs of noncoding sequence immediately upstream of the start codon. Disruption of the uORF 9 start codon, in the absence of simultaneous small 1-3 nucleotide deletions upstream had no effect on reporter gene expression. Similarly, small 1-3 nucleotide deletions upstream of the uORF in the absence of deleting the uORF start codon conferred a greater inhibitory effect on downstream gene expression. Thus, combined disruption of uORF 9, and the immediate upstream nucleotides is required to achieve increased protein expression.

In summary, we have demonstrated that disrupting two upstream open reading frames initiating translation 363 (uORF6; SEQ ID NO: 35) and 265 (uORF9; SEQ ID NO: 36) base pairs upstream of the BMPR2 CDS can increase BMPR2 protein expression by up to 250% across two cell lines. We have engineered ASO sequences targeting uORF 6 that increase endogenous BMPR2 expression across several cell lines, including primary cells from PAH patients. These sequences can be used to advantage to reduce vascular remodeling and/or fibrosis in target tissues.

We note that the degree of upregulation achieved by the mASO63 injection is roughly two-fold higher than that of wild-levels in healthy pulmonary arterial tissue. The threshold for pathological haploinsufficiency in the context of PAH lies between 50% of normal BMPR2 protein levels, and 100% in healthy, normal subjects. Blockade of BMPR2 uORF6 is therefore consistent with restoring haploinsufficient BMPR2 protein levels above those of the pathological threshold without reaching a supraphysiological level that might itself be problematic. We note that the higher hepatic expression of BMPR2 protein, and the relatively short time between ASO injection and tissue harvest did not reveal significant upregulation in liver. However, we fully expect that similar efficacy could be demonstrated for other disease contexts (cardiac fibrosis and hepatic fibrosis) by modifying the route of administration, dosage, and ASO nucleic acid chemistry used.

### REFERENCES

1. Fessel J P, Loyd J E, Austin E D. The genetics of pulmonary arterial hypertension in the post-BMPR2 era. Pulm Circ. 2011; 1: 305-319.
2. Evans J D W, Girerd B, Montani D, Wang X-J, Galie N, Austin E D, et al. BMPR2 mutations and survival in pulmonary arterial hypertension: an individual participant data meta-analysis. Lancet Respir Med. 2016; 4: 129-137.
3. Hamid R, Cogan J D, Hedges L K, Austin E, Phillips J A 3rd, Newman J H, et al. Penetrance of pulmonary arterial hypertension is modulated by the expression of normal BMPR2 allele. Hum Mutat. 2009; 30: 649-654.
4. Hopper R K, Moonen J-RAJ, Diebold I, Cao A, Rhodes C J, Tojais N F, et al. In Pulmonary Arterial Hypertension, Reduced BMPR2 Promotes Endothelial-to-Mesenchymal Transition via HMGA1 and Its Target Slug. Circulation. 2016; 133: 1783-1794.
5. Tian W, Jiang X, Sung Y K, Shuffle E, Wu T-H, Kao P N, et al. Phenotypically Silent Bone Morphogenetic Protein Receptor 2 Mutations Predispose Rats to Inflammation-Induced Pulmonary Arterial Hypertension by Enhancing the Risk for Neointimal Transformation. Circulation. 2019. pp. 1409-1425. doi:10.1161/circulationaha.119.040629
6. Atkinson C, Stewart S, Upton P D, Machado R, Thomson J R, Trembath R C, et al. Primary pulmonary hypertension is associated with reduced pulmonary vascular expression of type II bone morphogenetic protein receptor. Circulation. 2002; 105: 1672-1678.
7. Chen N-Y, D Collum S, Luo F, Weng T, Le T-T, M Hernandez A, et al. Macrophage bone morphogenic protein receptor 2 depletion in idiopathic pulmonary fibrosis and Group III pulmonary hypertension. Am J Physiol Lung Cell Mol Physiol. 2016; 311: L238-54.
8. Orriols M, Gomez-Puerto M C, Ten Dijke P. BMP type II receptor as a therapeutic target in pulmonary arterial hypertension. Cell Mol Life Sci. 2017; 74: 2979-2995.
9. Liang X-H, Shen W, Sun H, Migawa M T, Vickers T A, Crooke S T. Translation efficiency of mRNAs is increased by antisense oligonucleotides targeting upstream open reading frames. Nat Biotechnol. 2016; 34: 875-880.
10. Sasaki S, Sun R, Bui H-H, Crosby J R, Monia B P, Guo S. Steric Inhibition of 5′ UTR Regulatory Elements Results in Upregulation of Human CFTR. Molecular Therapy. 2019. pp. 1749-1757. doi:10.1016/j.ymthe.2019.06.016
11. Kearse M G, Wilusz J E. Non-AUG translation: a new start for protein synthesis in eukaryotes. Genes Dev. 2017; 31: 1717-1731.
12. Tirosh O, Cohen Y, Shitrit A, Shani O, Le-Trilling V T K, Trilling M, et al. The Transcription and Translation Landscapes during Human Cytomegalovirus Infection Reveal Novel Host-Pathogen Interactions. PLoS Pathog. 2015; 11: e1005288.
13. Pollard K S, Hubisz M J, Rosenbloom K R, Siepel A. Detection of nonneutral substitution rates on mammalian phylogenies. Genome Res. 2010; 20: 110-121.
14. Calvo S E, Pagliarini D J, Mootha V K. Upstream open reading frames cause widespread reduction of protein expression and are polymorphic among humans. Proc Natl Acad Sci USA. 2009; 106: 7507-7512.
15. Liang X-H, Sun H, Shen W, Wang S, Yao J, Migawa M T, et al. Antisense oligonucleotides targeting translation inhibitory elements in 5′ UTRs can selectively increase protein levels. Nucleic Acids Research. 2017. pp. 9528-9546. doi:10.1093/nar/gkx632
16. Conboy J. Antisense targeting of decoy exons can reduce intron retention and increase protein expression in human erythroblasts. doi:10.26226/morressier.5ebd45acffea6f735881b167
17. Lim K H, Han Z, Jeon H Y, Kach J, Jing E, Weyn-Vanhentenryck S, et al. Antisense oligonucleotide modulation of non-productive alternative splicing upregulates gene expression. Nat Commun. 2020; 11: 3501.
18. Johnstone T G, Bazzini A A, Giraldez A J. Upstream ORFS are prevalent translational repressors in vertebrates. EMBO J. 2016; 35: 706-723.
19. Lee D S M, Park J, Kromer A, Rader D J, Ritchie M D, Ghanem L R, et al. Disrupting upstream translation in mRNAs leads to loss-of-function associated with human disease. doi:10.1101/2020.09.09.287912
20. Vattem K M, Wek R C. Reinitiation involving upstream ORFs regulates ATF4 mRNA translation in mammalian cells. Proc Natl Acad Sci USA. 2004; 101: 11269-11274.
21. Lu P D, Harding H P, Ron D. Translation reinitiation at alternative open reading frames regulates gene expression in an integrated stress response. Journal of Cell Biology. 2004. pp. 27-33. doi:10.1083/jcb.200408003
22. Palam L R, Baird T D, Wek R C. Phosphorylation of eIF2 facilitates ribosomal bypass of an inhibitory upstream ORF to enhance CHOP translation. J Biol Chem. 2011; 286: 10939-10949.
23. Lee Y-Y, Cevallos R C, Jan E. An upstream open reading frame regulates translation of GADD34 during cellular stresses that induce eIF2alpha phosphorylation. J Biol Chem. 2009; 284: 6661-6673.
24. Reynolds A M, Holmes M D, Danilov S M, Reynolds P N. Targeted gene delivery of BMPR2 attenuates pulmonary hypertension. Eur Respir J. 2012; 39: 329-343.
25. Gaddipati R, West J D, Loyd J E, Blackwell T, Lane K A, Lane N M, et al. EGR1 is essential for transcriptional regulation of BMPR2. American Journal of Molecular Biology. 2011. pp. 131-139. doi:10.4236/ajmb.2011.13014
26. Aldred M A, Machado R D, James V, Morrell N W, Trembath R C. Characterization of the BMPR2 5′-untranslated region and a novel mutation in pulmonary hypertension. Am J Respir Crit Care Med. 2007; 176: 819-824.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A composition for enhancing expression of Bone morphogenetic protein receptor type II (BMPR2) comprising at least one antisense oligonucleotide (ASO).

2. The composition of claim 1, wherein said ASO is modified to increase stability in bodily fluids.

3. The composition of claim 1, wherein said ASO targets an upstream Open Reading Frame (uORF) and is selected from SEQ ID NO: 28, 30, 31, 32, 35, 36, and 38.

4. The composition of claim 1, wherein said ASO targets a sequence having 90% identity with one of the uORFs present in FIG. 1.

5. The composition of claim 1, wherein said ASO comprises one or more modifications which increase in vivo stability and or bioavailability.

6. The composition of claim 1, wherein said ASO targets at least one of uORF-6, said composition further comprising a biologically compatible carrier.

7. The composition of claim 1, wherein said ASO is present in a vector.

8. The composition of claim 1 which is formulated for administration selected from oral administration, aerosol administration, parenteral administration, intravenous administration or intracardiac administration.

9. The composition of claim 1, comprising wherein said ASOs are incorporated into a nanoparticle formulation suitable for aerosolized administration.

10. The composition of claim 1, comprising wherein said ASOs are incorporated into a formulation suitable for intracardiac administration.

11. A method of treating PAH associated pathophysiology in a subject in need thereof comprising administration of and effective amount of the composition of claim 1, said administration being effective to alleviate PAH symptoms in said subject.

12. The method of claim 11, wherein the fibrosis is pulmonary fibrosis, cardiac fibrosis, or liver fibrosis.

13. The method of claim 11, wherein the fibrosis is caused by pulmonary arterial fibrosis (PAH) and said treatment reduces vasoconstriction and vascular remodeling.

14. The method of claim 11, wherein the subject has PAH-related symptoms.

15. The method of claim 12, wherein the PAH-related symptoms are at least one of increased shortness of breath, fatigue, edema, swelling of the feet, legs and eventually the abdomen and neck, dizziness, fainting spells, and chest pain.

16. The method of claim 11, wherein at least one of the PAH-related symptoms improves.

17. The method of claim 11, further comprising administration of a supportive therapy.

18. The method of claim 17, wherein said supportive therapy is at least one of prostacyclins, Endothelin Receptor Antagonists (ERAs) Phosphodiesterase Inhibitors (PDE 5 Inhibitors) Selective IP Receptor Agonists, and Soluble Guanylate Cyclase (sGC) Stimulators Diuretics, Anticoagulants and Oxygen Therapy.

19. A method of modulating BMPR2 signalling in a target cell of interest, comprising administration of and effective amount of the composition of claim 1, said administration being effective to modulate BMPR2 signaling in said subject.


SEQUENCE LISTING (38 sequences)

SEQ ID NO: 1 - DNA, 20 bp, Artificial Sequence
  Firefly Forward
gacaccgctattctgagcgt

SEQ ID NO: 2 - DNA, 20 bp, Artificial Sequence
  Firefly reverse
ctcgaagcggtacatcagca

SEQ ID NO: 3 - DNA, 17 bp, Artificial Sequence
  Renilla Forward
cactgtctaaggtgggc

SEQ ID NO: 4 - DNA, 20 bp, Artificial Sequence
  Renilla Reverse
ggggtaatcagaatggcgct

SEQ ID NO: 5 - DNA, 23 bp, Artificial Sequence
  uORF1 Forward Primer
ctagctttctaggtggaatttac

SEQ ID NO: 6 - DNA, 21 bp, Artificial Sequence
  uORF2 Forward Primer
agcttgtccaaggaggcaggc

SEQ ID NO: 7 - DNA, 21 bp, Artificial Sequence
  uORF3 Forward Primer
taaggaatccagccttcccgg

SEQ ID NO: 8 - DNA, 21 bp, Artificial Sequence
  uORF4 Forward Primer
ccgcgggcgaagcgactaggg

SEQ ID NO: 9 - DNA, 26 bp, Artificial Sequence
  uORF5 Forward Primer
ctccgcacccaggatatgttttctcc

SEQ ID NO: 10 - DNA, 29 bp, Artificial Sequence
  uORF6 Forward Primer
accctggataagttttctcccagacctgg

SEQ ID NO: 11 - DNA, 24 bp, Artificial Sequence
  uORF7 Forward Primer
ggaaataattagggggatttcttc

SEQ ID NO: 12 - DNA, 21 bp, Artificial Sequence
  uORF8 Forward Primer
gatttcttctaggctccctgc

SEQ ID NO: 13 - DNA, 21 bp, Artificial Sequence
  uORF9 Forward Primer
cccacagacaagccttccgtt

SEQ ID NO: 14 - DNA, 21 bp, Artificial Sequence
  uORF10 Forward Primer
gccttccgttaggagggccgc

SEQ ID NO: 15 - DNA, 23 bp, Artificial Sequence
  Negative Control Forward Primer
gaactagttcagaccctcgcccc

SEQ ID NO: 16 - DNA, 21 bp, Artificial Sequence
  uORF1 Reverse Primer
cctatagtgagtcgtattaag

SEQ ID NO: 17 - DNA, 18 bp, Artificial Sequence
  uORF2 Reverse Primer
cacagtccccacgccttc

SEQ ID NO: 18 - DNA, 18 bp, Artificial Sequence
  uORF3 Reverse Primer
agtttcgcttcggtgctt

SEQ ID NO: 19 - DNA, 25 bp, Artificial Sequence
  uORF4 Reverse Primer
ctccgggaaggcaggattccttaag

SEQ ID NO: 20 - DNA, 18 bp, Artificial Sequence
  uORF5 Reverse Primer
ggcggggattcgatccgg

SEQ ID NO: 21 - DNA, 18 bp, Artificial Sequence
  uORF6 Reverse Primer
gcggagggcggggattcg

SEQ ID NO: 22 - DNA, 19 bp, Artificial Sequence
  uORF7 Reverse Primer
ctcgtagtttcacgatatc

SEQ ID NO: 23 - DNA, 18 bp, Artificial Sequence
  uORF8 Reverse Primer
ccccaaattatttccctc

SEQ ID NO: 24 - DNA, 20 bp, Artificial Sequence
  uORF9 Reverse Primer
gaaagcagggagccaagaag

SEQ ID NO: 25 - DNA, 21 bp, Artificial Sequence
  uORF10 Reverse Primer
atgtctgtggggaaagcaggg

SEQ ID NO: 26 - DNA, 18 bp, Artificial Sequence
  Negative Control Reverse Primer
ccgactgccgggaaggac

SEQ ID NO: 27 - RNA, 17 nt, Artificial Sequence
  ASO Control Sequence
cauggaggcggcuugag

SEQ ID NO: 28 - RNA, 18 nt, Artificial Sequence
  ASO 61
cauauccagggugcggag

SEQ ID NO: 29 - RNA, 14 nt, Artificial Sequence
  ASO 62-14
acauauccagggug

SEQ ID NO: 30 - RNA, 16 nt, Artificial Sequence
  ASO 62-16
  misc_feature (14)..(16): Said nucleotides are option modified iwth LNA chemistry
acauauccagggugcg

SEQ ID NO: 31 - RNA, 18 nt, Artificial Sequence
  ASO 62-18
acauauccagggugcgga

SEQ ID NO: 32 - RNA, 18 nt, Artificial Sequence
  ASO 63
aacauauccagggugcgg

SEQ ID NO: 33 - RNA, 18 nt, Artificial Sequence
  ASO 9
caugucuguggggaaagc

SEQ ID NO: 34 - RNA, 16 nt, Artificial Sequence
  ASO 62-mouse
acauauccagagugcc

SEQ ID NO: 35 - DNA, 57 bp, Homo sapiens
atgttttctcccagacctggatatttttttgatatcgtgaaactacgagggaaataa

SEQ ID NO: 36 - DNA, 39 bp, Homo sapiens
atgccttccgtttggagggccgcggcaccccgtccgagg

SEQ ID NO: 37 - DNA, 389 bp, Homo sapiens
tccccgccctccgcaccctggatatgttttctcccagacctggatatttttttgatatcgtgaaactacgagggaaataatttgggggatttcttcttggctccctgctttccccacagacatgccttccgtttggagggccgcggcaccccgtccgaggcgaaggaacccccccagccgcgagggagagaaatgaagggaatttctgcagcggcatgaaagctctgcagctaggtcctctcatcagccatttgtcctttcaaactgtattgtgatacgggcaggatcagtccacgggagagaagacgagcctcccggctgtttctccgccggtctacttcccatatttcttttctttgccctcctgattcttggctggcccagggatg

SEQ ID NO: 38 - DNA, 18 bp, Artificial Sequence
  mASO63
aacauauccagagugccg